(19) United States
(12) Patent Application Publication
(10) Pub. No.: US 2004/0180011 A1
(43) Pub. Date: Sep. 16, 2004

(54) Cosmetic formulation comprising alkyl phenyl silsesquioxane resins

(75) Inventor: Arndt Schlosser, Tecumseh, MI (US)

Correspondence Address: BROOKS KUSHMAN P.C., 1000 TOWN CENTER, TWENTY-SECOND FLOOR, SOUTHFIELD, MI, 48075, US

(73) Assignee: Wacker Chemical Corporation, Adrian, MI

(21) Appl. No.: 10/388,286

(22) Filed: Mar. 13, 2003

(51) Int. Cl. A61K 7/42; A61K 7/025; A61K 7/34; A61K 7/06; A61K 7/11

(52) U.S. Cl. 424/64; 424/66; 424/70.12; 424/59

(57) ABSTRACT

The use of C₂₋₂₀ alkyl phenyl silsesquioxane resins in cosmetic formulations provides cosmetics with good feel and water resistance. Lipsticks having excellent transfer resistance and high gloss may be prepared. Sunscreens and body lotions formulated with the resins exhibit improved UV absorbance. The resins may be supplied in flake form and are capable of melt blending.

### BACKGROUND OF THE INVENTION

1. Field of the Invention

The present invention pertains to cosmetic formulations which incorporate alkyl phenyl silsesquioxanes, and to a method for their preparation.

2. Background Art

Silicone resins are highly crosslinked organopolysiloxanes which are solid at room temperature and generally soluble in organic solvents. Reference may be had to Noll, CHEMISTRY AND TECHNOLOGY OF SILICONES, Chapter, 8, Academic Press, New York ©1968. Silicone resins may be classified into several groups depending upon the structural units of which they are composed, i.e. monofunctional (M) units, R₂SiO_{1/2}; difunctional (D) units, R₂SiO_{2/2}; trifunctional (T) units, RSiO_{3/2}; and tetrafunctional (Q) units, SiO_{4/2}.

Resins formed from M and T units are termed MT resins, while resins formed from M and Q units are termed MQ resins. Polymers formed substantially from M and D units are linear polymers or lightly branched polymers, and are not termed silicone resins by those skilled in the art. Resins formed only from T units are called T resins, or silsesquioxane resins.

Silicone resins of the MQ type have been widely used in cosmetic formulations, particularly where the organo (R) groups are all methyl groups. Such resins may be prepared, for example, by the hydrolysis of trimethylmethoxysilane (M) and tetramethoxysilane (Q) units. MT resins having all methyl groups may be prepared from cohydrolysis of trimethylmethoxysilane and methyltrimethoxysilane. MT resins having both methyl and phenyl groups may be prepared by cohydrolysis of trimethylmethoxysilane and phenyltrimethoxysilane.

Cosmetic powders such as face powder and rouge have used powdery organopolysiloxane resins to advantage. Such powdery resins may be used to produce powdered cosmetic formulations or creams containing solid resin particles which exhibit a smooth feeling when applied to the skin. Such powders require additional technological complexity in their manufacture, however, and often do not provide the requisite degree of hydrophobicity desired, particularly if the cosmetic is to be water resistant. JP 2000302878 exemplifies methyl phenyl silsesquioxane resin powders which are treated with a silanizing agent such as hexamethyldisilazane to react with residual silanol groups to render the particles hydrophobic. JP 2000345044 achieves a similar result by forming core/shell particles having mean diameters of 0.1 to 50 μm, the core/shell structure created by changing the ratios of methyl and phenyl silsesquioxane precursors during the second half of resin preparation.

In cosmetics which are liquid or semisolid, transfer resistance is desirable. Hydrophobic silicone resin powders cannot adequately provide this property. Rather, transfer resistance and other desirable properties are imparted by employing silicone resins in non-particulate form, i.e. melted into or dissolved in a cosmetic base composition. In the past, both methyl MQ resins and polymethylsilsesquioxane (methyl T resins) have been used for this purpose.

Methyl MQ resins have the distinct disadvantage that they have a high melting point. Thus, they must be dissolved in solvent if they are to be used in liquid or semisolid cosmetic formulations. Moreover, while methyl MQ resins increase transfer resistance in cosmetic formulations, their feel after application to the skin is in need of improvement, as is also the gloss associated with cosmetic formulations containing these resins.

### SUMMARY OF THE INVENTION

It has now been surprisingly discovered that silsesquioxane resins bearing both phenyl and C₂₋₂₀ alkyl groups may be formulated into liquid or semisolid cosmetic products which then offer excellent feel and high gloss, without sacrificing transfer resistance. The resins may be supplied in flake form, and may be melt blended or solution blended with the remaining cosmetic formulation ingredients.

### BRIEF DESCRIPTION OF THE DRAWINGS

FIG. 1 illustrates the improvement in gloss which may be obtained in lipsticks formulated with the alkyl phenyl silsesquioxane resins of the present invention;

FIG. 2 illustrates the increase in absorption of ultraviolet radiation in a hand protection lotion when employing an alkyl phenyl silsesquioxane resin of the present invention.

### DETAILED DESCRIPTION OF THE PREFERRED EMBODIMENT(S)

The cosmetic formulations which are the subject of the invention are liquid and semisolid cosmetic products. By semisolid is meant a consistency which is a relatively soft solid, e.g. of a waxy nature. Examples include lipstick and mascara. By liquid is meant creams, lotions, etc. Suitable cosmetic formulations include sunscreens, foundations, body lotions, and hair care products, in addition to those listed above. This list is non-exclusive.

The silicone resins useful herein are silsesquioxane resins which bear phenyl and C₂₋₂₀ alkyl groups. These resins hereafter are termed “alkyl phenyl silsesquioxane” resins. It is noted that the alkyl groups are C₂₋₂₀ alkyl or cycloalkyl groups, i.e. methyl phenyl resins are not within this definition.

In addition to the foregoing constituents, the resins may also bear methyl groups in addition to the “higher” alkyl and phenyl groups. The preferred resins are exclusively T resins, i.e. silsesquioxane resins per se. However, it would not depart from the spirit of the invention to include minor amounts, i.e. preferably less than 10 mol percent each of M, D, and Q units, although higher amounts are possible, provided that these resins are capable of melt blending or solution blending into the cosmetic formulation.

Thus, the preferred resins are those of the formula

[RSiO_{3/2}]ₐ[R¹SiO_{3/2}]_{b}[R²SiO_{3/2}]_{c}[R₃³SiO_{1/2}]_{d}[R_{2[}³SiO_{2/2}]ₑ[SiO_{4/2}]_{f}

where R is methyl; R¹ is C₂₋₂₀ alkyl or C₅₋₂₀ cycloalkyl, preferably, C₃₋₈ alkyl or C₆₋₁₂ cycloalkyl; R² is phenyl, R³ is C₁₋₂₀ alkyl, C₅₋₂₀ cycloalkyl, C₇₋₁₄ aralkyl, C₇₋₁₄ alkaryl, or C₆₋₁₀ aryl, preferably C₁₋₈ alkyl, C₆₋₁₂ cycloalkyl, or C₆ aryl; and a, b, and c are such that their respective siloxy groups preferably comprise at least 90 mol percent of the total of groups a-f, and at least one R¹ and at least one R² is present. By “group a” or “moiety a” is meant the siloxy group of which “a” is the substituent.

Preferably, b and c are such that the R¹ “higher” alkyl and R² (phenyl) groups are present in from 20 to 100 mol percent of all siloxy groups, more preferably 50 to 100 mol percent. The number of methyl silsesquioxy groups RSiO_{3/2} is preferably less than 30 mol percent, more preferably less than 20 mol percent. C₂₋₂₀ alkyl groups and phenyl groups are preferably present in a mol ratio of 1:10 to 10:1. The products are preferably produced in flake form, and are preferably not hydrophobicized (silylated). In addition to the siloxy units described above, the resins may also contain surfactant groups, for example polyoxyalkylene groups, preferably polyoxyethylene groups, glycosidyl groups, etc., although these are not preferred.

The alkyl phenyl silsesquioxane resins may be prepared by standard techniques known to the industry. For example, the resins may be prepared by cohydrolysis of mixtures of C₂₋₂₀ alkyl trialkoxy silanes and phenyl trialkoxy silanes, optionally in admixture with methyltrialkoxy silanes. When minor amounts of M, D, or Q units are desired, these may be supplied in the form of their respective alkoxysilane precursors. It is desired that the products contain less than 30 mol percent of M, D, and Q units, and preferably are substantially or wholly free of such units. In lieu of alkoxy silane precursors, other hydrolyzable precursors such as halosilanes, acetoxysilanes, and the like may be used. Reference may be had to Chapter 8 of Noll, CHEMISTRY AND TECHNOLOGY OF SILICONES. The alkyl phenyl silsesquioxane resins preferably soften in the range of 30° C. to 90° C., more preferably 30° C. to about 70° C., and most preferably in the range of 40-60° C., as determined by DIN 53180 “Softening Point of Resins.” A preferred resin is Wacker Belsil® SPR 45, available from Wacker Chemical, Adrian, Mich., a propyl and phenyl silsesquioxane resin having a ratio of propyl to phenyl groups of about 1:3, and a softening point in the range of 40-50° C.

Formulation of cosmetics employing the alkyl phenyl silsesquioxane resins of the present invention can be performed by those skilled in the art. Depending upon the particular cosmetic, the amount of the alkyl phenyl silsesquioxane resin may range from about 0.1 weight percent to about 50 weight percent, but is typically within the range of 0.5 weight percent to about 20 weight percent, more preferably 1 weight percent to 10 weight percent. The alkyl phenyl silsesquioxane resin may also be used in conjunction with other silicone resins, in particular methyl MQ resins (trimethylsiloxysilicate) and methyl T resins (polymethylsilsesquioxane). The alkyl phenyl silsesquioxane resins may also be used in conjunction with organopolysiloxane powders prepared by hydrosilylative addition polymerization, as disclosed, for example, in U.S. Pat. Nos. 6,423,322; 5,760,116; and European published application EP 113 24 30 A1. It is expected that such powders will remain in particulate form in the finished cosmetic formulation.

The alkyl phenyl silsesquioxane resins may be melt or solution blended with remaining formulation ingredients. “Melt blending” is used in the conventional sense, e.g. to mean that the silicone resin is melted and added to liquid or solid ingredients to form a liquid or semisolid product, or is added as a solid to a liquid or semisolid product maintained at a temperature such that the resin melts or dissolves into the other ingredients without the use of a solvent. By solution blending is meant employing a solvent in which the resin is soluble. The solvent may be used to prepare a solution of the resin which is subsequently added to other ingredients, or the resin may be added to a composition of solvent containing other ingredients, in either case the solvent facilitating solution, in whole, or in part, of the resin. The solvent is generally removed from the cosmetic formulation. Aromatic solvents, aliphatic solvents, and low viscosity silicone oils and cyclic silicones may be used as solvents, as may also various alcohols, ketones, ethers, and the like. Aromatic solvents are generally avoided in cosmetic formulation preparation, however, and thus if solution blending is to be used, the alkyl phenyl silsesquioxane resin must be soluble to the desired extent in cosmetically acceptable solvents. Preferably, the silicone resin is melt blended.

The cosmetic formulations generally contain customary additional ingredients such as but not limited to dyes, pigments, fillers, sunscreens, viscosifiers, thixotropes, emollients, moisturizers, waxes, surfactants, plasticizers, perfumes, astringents, deodorants, and the like. Examples which are typical are presented herein. Many of the formulations contain substantial amounts of water. The formulations, with the exception of insoluble ingredients such as fillers and pigments, may constitute a solution, a dispersion, or an emulsion. Emulsions of both the water-in-oil and oil-in-water type are contemplated, as are complex multiple emulsions such as oil-in-water-in-oil and water-in-oil-in-water.

The cosmetic formulations may be prepared by standard techniques known to the skilled artisan in the cosmetics industry. In some instances, all ingredients may be added to a heated mixer and blended at low or high shear, depending upon the particular formulation, and then cooled once the desired homogeneity is obtained. Heat may sometimes be dispensed with if all ingredients are mutually soluble, for example in many sunscreen preparations where considerable oily ingredients are present and water is substantially absent. However, heating may facilitate more rapid mixing.

Generally, however, many formulations are prepared as separate “phases” which are then blended together. Some phases may require heating to melt blend the various ingredients, e.g. silicone resins, fatty substances, waxes, etc., while other phases may require simple mixing. The phases are then combined with the appropriate type of agitation and, if required, heating. In the case of emulsions and dispersions, a surfactant is often necessary, and mixing may require high shear, for example using rotor/stator mixers, high speed turbine mixers, three roll mills, etc.

For semisolid products, especially those containing polyolefin waxes, beeswax, or other natural or synthetic waxy substances, it may be desirable to continue mixing during cooling of a melt blend to avoid precipitation of large crystals and thus to provide a more homogenous and smooth-feeling product. All these techniques are well known to the skilled cosmetic formulator.

Having generally described this invention, a further understanding can be obtained by reference to certain specific examples which are provided herein for purposes of illustration only and are not intended to be limiting unless otherwise specified.

### EXAMPLE 1

A lipstick formulation was prepared from the following ingredients and phases presented in Table 1:

TABLE 1

| Ingredient | Parts |
|---|---|
| Phase A | |
| Isododecane | 10.00% |
| IsopropylMyristate | 12.00% |
| Phase B | |
| Ariabel Sienna | 3.50% |
| D&C red No. 7 Ca Lake | 1.00% |
| Titanium Dioxide Cl77892 | 3.5% |
| Phase C | |
| Carnauba wax | 8.50% |
| Ceteth-2 | 3.50% |
| White Beeswax | 5.50% |
| Microcrystalline Wax | 6.00% |
| Polyethylene | 3.00% |
| Phase D | |
| Wacker-Belsil ® CM 040¹ | 21.30% |
| Wacker-Belsil ® DM 1¹ plus | 4.50% |
| Wacker-Belsil ® SPR 45¹ | 10.00% |
| Phase E | |
| Talc | 3.50% |
| Phase F | |
| Tocopheryl Acetate | 0.20% |
| Phase G | |
| Mica Cl77891 | 4.00% |

¹Trademarks of Wacker-Chemie GmbH

Phase C was melt blended and combined with phases A and B. Phases D, E, F, and G were then added, mixed to homogeneity, and the mixture poured into molds to solidify.

### COMPARATIVE EXAMPLE C1

A lipstick formulation was prepared in accordance with Example 1, but the Wacker Belsil® SPR alkyl phenyl resin was substituted by the same quantity of Wacker Belsil® TMS, a methyl MQ resin.

Transfer resistance of the lipsticks of Example 1 and Comparative Example 1 were assessed by applying uniform layers on clean paper cards, juxtaposing a clean card atop the coated card, and rolling a weight over the clean card. The color change of the coating is measured using an X-rite 948 calorimeter. Color as ΔE was calculated from the L*a*b* values as follows:

ΔE=[(ΔL*)²+(Δa*)²+(Δb*)²]^{1/2}. Greater ΔE corresponds to increased transfer.

After 50 blots, the two samples demonstrated the same transfer resistance, while after 60 blots, the transfer resistance of the Example 1 formulation showed a transfer resistance of 0.97 relative to that of Comparative Example 1. Both Examples exhibited high transfer resistance, much higher than lipstick containing no silicone resin.

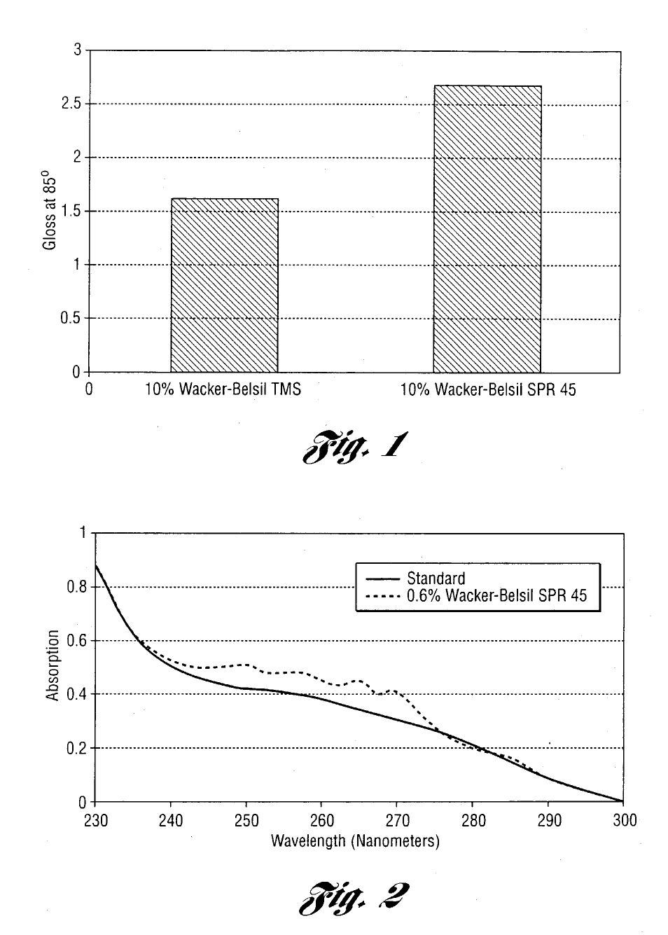

Gloss of the lipstick formulations was measured by a Hunter Pro Gloss 3 at 85° on lipstick applied to a paper card. Despite the fact that both formulations employed silicone resins, the alkyl phenyl silsesquioxane resin-containing formulation of Example 1 exhibited much higher gloss, as shown in FIG. 1. The gloss was about 67% higher when the subject invention alkyl phenyl silsesquioxane resin was employed.

### EXAMPLE 2

A hand protection lotion was formulated with Wacker Belsil® SPR 45. The formulation is presented in Table 2 below. The Phases A and B ingredients were heated to 80° C. and A added to B with vigorous agitation.

TABLE 2

| Ingredient | Parts |
|---|---|
| Phase A | |
| Cetyl Alcohol | 0.65% |
| Wacker-Belsil SPR 45² | 0.60% |
| Wacker-Belsil ® DM 35² | 0.70% |
| Wacker-Belsil ® DM 200² | 1.10% |
| Isopropyl Myristate | 2.35% |
| Lanolin Acid | 0.65% |
| PEG-12 | 2.35% |
| Stearic Acid | 3.50% |
| Phase B | |
| Triethanolamine | 0.65% |
| Aqua | 87.45% |

²Trademarks of Wacker-Chemie GmbH

### COMPARATIVE EXAMPLE C2

A body lotion formulation was prepared as in Example 2, but without the use of 0.6 weight percent Wacker-Belsil® SPR 45.

The UV absorption spectrum of diluted samples of the body lotions of Example 2 and Comparative Example C2 were measured and compared. The results are presented in FIG. 2. Despite constituting only 0.6 weight percent of the formulation, the lotion of Example 2 exhibited about a 10% increase in UV absorption over the range of 250 nm to 370 nm, even in the diluted samples.

### EXAMPLE 3

Wacker-Belsil® SPR 45 was employed to prepare a make-up foundation in accordance with the following formulation. The phases were prepared separately and blended together in order. Phase C was prepared by melt blending the ingredients. The foundation had excellent feel to the skin and demonstrated excellent water resistance.

TABLE 3

| Ingredient | Parts |
|---|---|
| Phase A | |
| Lanette N | 5.00% |
| Isopropyl Myristate | 8.00% |
| Isohexadecane | 5.00% |
| Phase B | |
| Glycerin | 3.00% |
| Xanthan Gum | 0.40% |
| Aqua | 52.2% |
| Phase C | |
| Pigments | 4.30% |
| Talc | 2.50% |
| Wacker-Belsil ® CM040³ | 8.00% |
| Wacker-Belsil ® SPR³ | 5.00% |
| Wacker-Belsil ® LDM 3107³ | 4.00% |
| Wacker-Belsil ® PDM 1000³ | 2.00% |
| Phase D | |
| Euxyl K 300 | 1.20% |
| Perfume | 1.20% |

³Trademarks of Wacker-Chemie GmbH

### EXAMPLE 4

A mascara formulation was prepared by separately preparing the phases A, B, and C as presented in Table 4, and blending together. Upon cooling, a semisolid mascara is produced.

TABLE 4

| Ingredient | Parts |
|---|---|
| Phase A | |
| Acrylates/Octylacrylamide | 5.00% |
| Copolymer | |
| Methylparabene | 0.20% |
| Propylene Glycol | 3.00% |
| Triethanolamine | 3.10% |
| Imidazolidinyl Urea | 0.15% |
| Aqua | 49.95% |
| Phase B | |
| Candelilla wax | 4.50% |
| Carnauba wax | 1.00% |
| Cetyl Alcohol | 3.00% |
| Dimethiconol Beeswax | 5.50% |
| Ozokerite | 2.00% |
| Propylparaben | 0.10% |
| Stearic Acid | 5.00% |
| Wacker-Belsil ® TMS 803⁴ | 5.00% |
| Phase C | |
| Wacker-Belsil ® SPR 45⁴ | 1.50% |
| Iron Oxides | 11.00% |

⁴Trademarks of Wacker-Chemie GmbH

### EXAMPLE 5

A waterproof mascara formulation was prepared from the ingredients listed in Table 5.

TABLE 5

| Ingredient | Parts |
|---|---|
| Phase A | |
| PVP/Eicosene Copolymer | 8.00% |
| Sorbitan Isostearate; PEG- | 3.50% |
| 2 Hydrogenated Castor Oil; | |
| Ozokerite, Hydrogenated Castor | |
| PEG-7 Hydrogenated Castor Oil | 0.50% |
| White Beeswax | 7.50% |
| Mineral Oil | 15.00% |
| Methylparaben | 0.20% |
| Wacker HDK H 15⁵ | 1.00% |
| Wacker-Belsil ® SPR 45⁵ | 2.00% |
| Phase B | |
| CI 77499, Dimethicone | 10.00% |
| Phase C | |
| Wacker-Belsil ® CM 040⁵ | 7.00% |
| Wacker-Belsil ® CM 1000⁵ | 7.00% |
| Phase D | |
| Disodium EDTA | 0.05% |
| Sodium Dehydroacetate | 0.15% |
| Aqua | 25.65% |
| Phase E | |
| Diazolidinyl Urea | 0.25% |
| Aqua | 2.00% |
| Phase F | |
| Polviol G 18/140⁵ (10% sol.) | 10.00% |
| Phase G | |
| Perfume | 0.20% |

⁵Trademarks of Wacker-Chemie GmbH

The phase A ingredients were melted. Phase B was added in small increments to phase C while homogenizing. Phases B and C (admixture) were homogenized into phase A, following which phase D, at 65-70° C., was added. The homogenous mixture was cooled to 40° C. with agitation, then phases E, F, and G were added and homogenized for about 5 minutes after each addition. The product was cooled to room temperature with continuous mixing.

### EXAMPLE 6

A sunscreen formulation is created by preparing phases A, B, C, and D as set forth in Table 6 and blending together. A liquid formulation is produced.

TABLE 6

| Ingredient | Parts |
|---|---|
| Phase A | |
| Cetyl Alcohol | 1.00% |
| Benzophenone-3 | 2.50% |
| Isopropyl Dibenzoylmethane | 5.00% |
| Isopropyl Myristate | 1.00% |
| PVP/PA Copolymer | 2.00% |
| Stearic Acid | 6.00% |
| Wacker-Belsil ® DM 350⁶ | 3.30% |
| Phase B | |
| Hydroxyethylcellulose | 0.50% |
| Aqua | 60.00% |
| Phase C | |
| Triethanolamine | 2.50% |
| Wacker-Belsil ® SPR 45⁶ | 3.00% |
| Wacker-Belsil ® CM 040⁶ | 13.00% |
| Phase D | |
| DMDM Hydantoin | 0.20% |

⁶Trademarks of Wacker-Chemie GmbH

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. In a cosmetic formulation suitable for application to the skin, the improvement comprising the incorporation of from 0.1 weight percent to about 50 weight percent, based on the total weight of the cosmetic formulation, of at least one alkyl phenyl silsesquioxane resin comprising moieties
[RSiO_{3/2}]ₐ[R¹SiO_{3/2}]_{b}[R²SiO_{3/2}]_{c}[R₃³SiO_{1/2}]_{d}[R_{2[}³SiO_{2/2}]ₑ[SiO_{4/2}]_{f}
where R is methyl; R¹ is C₂₋₂₀ alkyl or C₅₋₂₀ cycloalkyl; R² is phenyl, R³ is C₁₋₂₀ alkyl, C₅₋₂₀ cycloalkyl, C₇₋₁₄ aralkyl, C₇₋₁₄ alkaryl, or C₆₋₁₀ aryl; and d, e, and f are such that their respective moieties comprise in total less than 30 mol percent of all of the moieties a)-f).

2. The cosmetic formulation of claim 1, wherein R¹ is C₃₋₈ alkyl.

3. The cosmetic formulation of claim 1, wherein d, e, and f are zero.

4. The cosmetic formulation of claim 1, wherein said alkyl phenyl silsesquioxane softens in the range of 30° C. to 70° C.

5. The cosmetic formulation of claim 1, which is selected from the group consisting of lipsticks, mascaras, foundations, body lotions, and sunscreens.

6. The cosmetic formulation of claim 1, which is a lipstick.

7. The cosmetic formulation of claim 1, wherein the moieties which correspond to b and c comprise 20 to 100 mol percent of said alkyl phenyl silsesquioxane.

8. The cosmetic formulation of claim 7, wherein d, e, and f are zero.

9. The cosmetic formulation of claim 1, wherein d is zero.

10. A process for the preparation of a cosmetic formulation of claim 1, comprising blending said alkyl phenyl silsesquioxane with additional cosmetic formulation ingredients.

11. The process of claim 10, wherein said blending takes place at a temperature within a softening range of said alkyl phenyl silsesquioxane.

12. The process of claim 10, wherein said alkyl phenyl silsesquioxane is dissolved in a solvent to form an alkyl phenyl silsesquioxane solution, and the solution is blended with other cosmetic formulation ingredients.

13. The process of claim 12, further comprising removing at least a portion of said solvent following blending.

14. The process of claim 10, wherein the cosmetic formulation is selected from the group consisting of lipsticks, mascaras, foundations, body lotions, and sunscreens.

15. The cosmetic formulation of claim 10, wherein R¹ is C₃₋₈ alkyl.

16. The cosmetic formulation of claim 10, wherein d, e, and f are zero.

17. The cosmetic formulation of claim 10, wherein said alkyl phenyl silsesquioxane softens in the range of 30° C. to 70° C.

18. The cosmetic formulation of claim 10, wherein the moieties which correspond to b and c comprise 20 to 100 mol percent of said alkyl phenyl silsesquioxane.

19. The cosmetic formulation of claim 10, wherein d is zero.
